(19) United States
(12) Patent Application Publication
(10) Pub. No.: US 2004/0176699 A1
(43) Pub. Date: Sep. 9, 2004

(54) Thermography catheter with improved wall contact

(75) Inventors: Blair Walker, Mission Viejo, CA (US); Tracy Maahs, Rancho Santa Margarita, CA (US); Jesus Flores, Perris, CA (US)

Correspondence Address: STRADLING YOCCO CARLSON & RAUTH, SUITE 1600, 660 NEWPORT CENTER DRIVE, P.O. BOX 7680, NEWPORT BEACH, CA, 92660, US

(73) Assignee: Volcano Therapeutics, Inc.

(21) Appl. No.: 10/378,249

(22) Filed: Mar. 3, 2003

(51) Int. Cl. A61B 5/00

(52) U.S. Cl. 600/549; 600/474; 600/467; 600/309; 600/547

(57) ABSTRACT

A flexible thermography catheter which includes an elongated body having a proximal end, a distal end, and a distal section, an expandable body comprising at least one independently movable support arm located at the distal section of the elongated body, at least one support arm retainer positioned on the distal section of the elongated body, and at least one sensor positioned on at least one of the distal section, the expandable body, and the at least one support arm. The at least one support arm retainer is configured to independently secure at least one section of the at least one support arm to the distal section of the elongated body in independently movable relation to each of the at least one support arm and the elongated body.

### BACKGROUND

Recent studies have suggested that the rupture of vulnerable plaque within a blood vessel may trigger 60% to 70% of fatal myocardial infarctions. Moreover, vulnerable plaque erosion or ulceration may trigger 25% to 30% of fatal infarctions. The ability to detect and locate vulnerable plaque within a blood vessel may permit the treatment of the area prior to the rupture, ulceration, or erosion of the plaque deposit. However, vulnerable plaques are often undetectable using conventional techniques such as angiography. Indeed, the majority of vulnerable plaques that led to an infarction in the recent studies occurred in coronary arteries that appeared normal or only mildly stenotic on angiograms performed prior to the infarction.

Studies of the composition of vulnerable plaque suggest that the presence of inflammatory cells, particularly in a plaque having a large lipid core, may be a powerful predictor of ulceration and/or imminent plaque rupture. For example, in plaque erosion, the endothelium beneath a thrombus formed on the interior wall of a blood vessel may be replaced by or interspersed with inflammatory cells. Further, recent literature suggests that the presence of inflammatory cells within vulnerable plaque may be identified by detecting thermal discontinuities associated with the metabolic activity of these inflammatory cells, thereby permitting the identification and detection of vulnerable plaque. Moreover, it is generally known that activated inflammatory cells have a heat signature that differs from that of connective tissue cells.

In light of the foregoing, it is believed that one way of detecting whether specific plaque is vulnerable to rupture and/or ulceration involves measuring the temperature of the walls of arteries in the region of a deposit of plaque. Once vulnerable plaque is identified, localized treatments may be developed to specifically address the problems. These treatments may include, for example, the localized delivery of therapeutic drugs to the area or thermal therapy.

Several devices capable of examining the thermal characteristics of vulnerable plaque have been developed. For example, the commonly assigned U.S. Pat. No. 6,245,026, issued on Jun. 12, 2001, which is incorporated by reference in its entirety herein, describes a number of thermography devices capable of being introduced into a blood vessel of a patient, advanced to a area of interest within the body of a patient, and used to examine the thermal characteristics of plaque deposits. In addition, alternate thermography catheters are described in U.S. Pat. No. 5,871,449, issued on Feb. 16, 1999, U.S. Pat. No. 5,935,075, issued on Aug. 10, 1999, and U.S. Pat. No. 5,924,997, issued on Jul. 20, 1999, each of which are incorporated by reference in their entirety herein.

In light of the foregoing, there is an ongoing need for a low profile, flexible thermography catheter which is capable of transversing a tortuous pathway in vivo, and detecting, locating, and treating inflamed or vulnerable plaque and/or areas of thermal discontinuity within the blood vessels of a patient.

### SUMMARY

A flexible thermography catheter for detecting, locating, and treating inflamed or vulnerable plaque in vivo is disclosed. More specifically, the flexible thermography catheter is capable of traversing a tortuous pathway while offering improved vessel wall contact over prior art systems.

In one embodiment, the thermography catheter comprises an elongated body having a proximal end, a distal end and a distal section, a deployable expandable body disposed on the distal section of the elongated body having at least two support arms configured to be radially displaceable from a longitudinal axis of the expandable body and independently movable in a radial direction in relation to each other when deployed and having a proximal section, a distal section, and a medial section configured to be radially displaceable a greater distance from the longitudinal axis than the proximal and distal sections, and a thermal sensor disposed on the medial section of at least one of the support arms and configured to contact the body vessel when deployed.

In another embodiment, the catheter comprises an elongated body having a proximal end, a distal end and a distal section, an expandable body located at the distal section of the elongated body and comprising at least one support arm configured to be radially displaceable from and independently movable in relation to a longitudinal axis L of the expandable body and another support arm, the at least one support arm having a proximal section configured to be radially displaceable a distance D1 from the longitudinal axis L, a medial section configured to be radially displaceable a distance D2 from the longitudinal axis L, and a distal section configured to be radially displaceable a distance D3 from the longitudinal axis L, wherein distance D2 is greater than distances D1 and D3, and at least one sensor positioned on at least one of the proximal section of the at least one support arm, the medial section of the at least one support arm, the distal section of the at least one support arm, the expandable body, and the elongated body.

In an alternate embodiment, the catheter may comprise an elongated body having a proximal end, a distal end and a distal section, an expandable body located at the distal section of the elongated body and comprising at least one support arm, at least one support arm retainer positioned on the distal section of the elongated body, the at least one support arm retainer independently securing at least one section of the at least one support arm to the elongated body in independently movable relation to another support arm and the elongated body, and at least one sensor positioned on at least one of the distal section, the expandable body, and the at least one support arm.

In yet another embodiment, the catheter comprises an elongated body having a proximal end, a distal end and a distal section and having an inner body member positioned within a retractable outer sleeve, an expandable body located at the distal section of the elongated body and positionable within the outer sleeve and comprising at least one support arm coupled proximately to the elongated body in fixed relation, at least one support arm retainer positioned on the distal section of the elongated body and independently coupling a distal section of the at least one support arm to the distal section of the elongated body in independently movable relation to each of the at least one support arm, and at least one thermal sensor positioned on at least one of the distal section, the expandable body, the deployment support member, and the at least one support arm.

An alternate embodiment of the thermography catheter is disclosed and includes an elongated body having a distal section, an expandable body defining an expandable body passage disposed on the distal section of the elongated body, at least one thermal sensor configured to contact a vessel wall disposed on the expandable body, and an inner body having a flexible distal section co-linear with the expandable body.

In addition, an expandable body for measuring one or more thermal characteristics of a vessel is disclosed. The expandable body may comprise at least two deployable support arms configured to be radially displaceable from a longitudinal axis of the expandable body and independently movable in relation to each other when deployed, the at least two support arms having a proximal section configured to be radially displaceable a distance D1 from the longitudinal axis L, a medial section configured to be radially displaceable a distance D2 from the longitudinal axis L, and a distal section configured to be radially displaceable a distance D3 from the longitudinal axis L, wherein distance D2 is greater than distances D1 and D3, and at least one sensor positioned on the medial section of at least one of the at least two support arms.

In another embodiment, the expandable body may comprise at least one support arm configured to be radially displaceable from and independently movable in relation to a longitudinal axis L of the expandable body and another support arm and having a proximal section configured to be radially displaceable a distance D1 from the longitudinal axis L, a medial section configured to be radially displaceable a distance D2 from the longitudinal axis L, and a distal section configured to be radially displaceable a distance D3 from the longitudinal axis L, wherein distance D2 is greater than distances D1 and D3. At least one sensor may be positioned on at least one of the proximal section of the at least one support arm, the medial section of the at least one support arm, the distal section of the at least one support arm, and the expandable body.

In yet another embodiment, the expandable body may comprise at least one support arm defining the expandable body, at least one support arm retainer configured to retain at least a section of the at least one support arm therein in independently movable relation to at least another support arm, and at least one sensor positioned on at least one of the at least one support arm and the at least one support arm retainer.

In an alternate embodiment, the expandable body may comprise at least one support arm defining the expandable body and having a sensor channel formed thereon, at least one support arm retainer configured to retain at least a section of the at least one support arm therein in independently movable relation to at least another support arm, and at least one sensor positioned within the sensor channel of the at least one support arm.

In yet another embodiment, the expandable body may comprise at least one support arm defining an expandable body having a longitudinal axis L and having a first relaxed state displaced a first distance D1 from the longitudinal axis L and a second compressed state displaced a second distance D2 from the longitudinal axis L wherein the first distance D1 is greater than the second distance D2, at least one support arm retainer configured to retain at least a section of the at least one support arm therein in independently movable relation to at least another support arm, and at least one sensor positioned on at least one of the at least one support arm and the at least one support arm retainer.

In another embodiment, the expandable body may comprise at least two support arms configured to be radially displaceable from a longitudinal axis of the expandable body and independently movable in a radial direction in relation to each other when deployed and having a proximal section, a distal section, and a medial section configured to be radially displaceable a greater distance from the longitudinal axis than the proximal and distal sections, a distal tip positioned on a distal portion of the expandable body, and a thermal sensor disposed on the medial section of at least one of the support arms and configured to contact the body vessel when deployed.

### BRIEF DESCRIPTION OF THE DRAWINGS

Various embodiments of a flexible thermography catheter will be explained in more detail by way of the accompanying drawings, wherein components having similar but not necessarily the same or identical features, may have the same reference numeral, and wherein:

FIG. 1 shows a perspective view of an embodiment of a thermography catheter wherein the expandable body is in a non-deployed state;

FIG. 2 shows a perspective view of the thermography catheter wherein the expandable body is in a deployed state;

FIG. 3 shows an enlarge perspective view of the of a handle of a thermography catheter of FIG. 1;

FIG. 4 shows an elevational view of a distal section of the elongated body of the thermography catheter of FIG. 1;

FIG. 5 shows a transverse cross-sectional view of the elongated body of the thermography catheter as taken along the lines 5-5 of FIG. 4;

FIG. 6 shows an elevational view in partial section of the expandable body of the thermography catheter in a non-deployed state;

FIG. 7 shows an elevational view of a distal section of an elongated body of an alternative embodiment of a thermography catheter;

FIG. 8 shows an elevational view in partial section of the thermography catheter of FIG. 7;

FIG. 9 shows a perspective view of a distal section of the elongated body of the thermography catheter shown in FIG. 7;

FIG. 10 shows of an expandable body of an alternative embodiment of a thermography catheter in a non-deployed state;

FIG. 11 shows an elevational view in partial section of the expandable body of the thermography catheter of FIG. 7 in a non-deployed state;

FIG. 12 shows a perspective view of the expandable body of FIG. 11 in an expanded state;

FIG. 13 shows a perspective view of an embodiment of a support arm retainer of a thermography catheter;

FIG. 14 shows a perspective view of an embodiment of a support arm retainer of a thermography catheter;

FIG. 15 shows a perspective view of an embodiment of an expandable body of a thermography catheter;

FIG. 16 shows a perspective view of an embodiment of a support arm of a thermography catheter;

FIG. 17 shows a perspective view of an embodiment of a support arm of a thermography catheter;

FIG. 18 shows a perspective view of an embodiment of a support arm of a thermography catheter;

FIG. 19 shows a perspective view of an embodiment of a support arm of a thermography catheter;

FIG. 20 is a perspective view of an embodiment of a support arm of a thermography catheter;

FIG. 21 is a perspective view of an embodiment of a support arm of a thermography catheter;

FIG. 22 shows a perspective view of an embodiment of an expandable body of a thermography catheter;

FIG. 23 shows a perspective view of an embodiment of an expandable body of a thermography catheter positioned within a blood vessel;

FIG. 24 is an elevational top view of an embodiment of a support arm of a thermography catheter;

FIG. 25 is an elevational view of the support arm shown in FIG. 24;

FIG. 26 shows a perspective view of another embodiment of a support arm of a thermography catheter;

FIG. 27 is an elevational view of the support arm shown in FIG. 26;

FIG. 28 shows a perspective view of an embodiment of a sensor prior to being coupled to a support arm of a thermography catheter;

FIG. 29 shows a perspective view of an embodiment of a sensor positioned within a sensor channel formed on a support arm of a thermography catheter;

FIG. 30 is an elevational view of an embodiment of a sensor disposed on a support arm of a thermography catheter;

FIG. 31 shows a longitudinal transverse cross sectional view of a support arm as viewed along the lines 31-31 shown in FIG. 30;

FIG. 32 shows a perspective view of an embodiment of an expandable body of a thermography catheter in a deployed state;

FIG. 33 shows a side view of an embodiment of a sensor disposed on a support arm of a thermography catheter positioned in a vessel with the sensor in contact with the vessel wall;

FIG. 34 shows a an elevational view of embodiment of an expandable body of a thermography catheter;

FIG. 35 shows an elevational view of an embodiment of an expandable body of a thermography catheter;

FIG. 36 shows an elevational view of an embodiment of an expandable body of a thermography catheter;

FIG. 37 shows an elevational view of an embodiment of an expandable body of a thermography catheter.

FIG. 38 shows an inner assembly of a thermography catheter;

FIG. 39 shows an outer assembly of a thermography catheter;

FIG. 40 shows the inner assembly of a thermography catheter nested within the outer assembly;

FIG. 41 shows the proximal end of the outer assembly with luer adaptor an a dynamic seal for flushing;

FIG. 42 shows a longitudinal cross-section of the proximal end of the outer assembly of FIG. 41 taken through section line A-A.

### DETAILED DESCRIPTION OF THE DRAWINGS

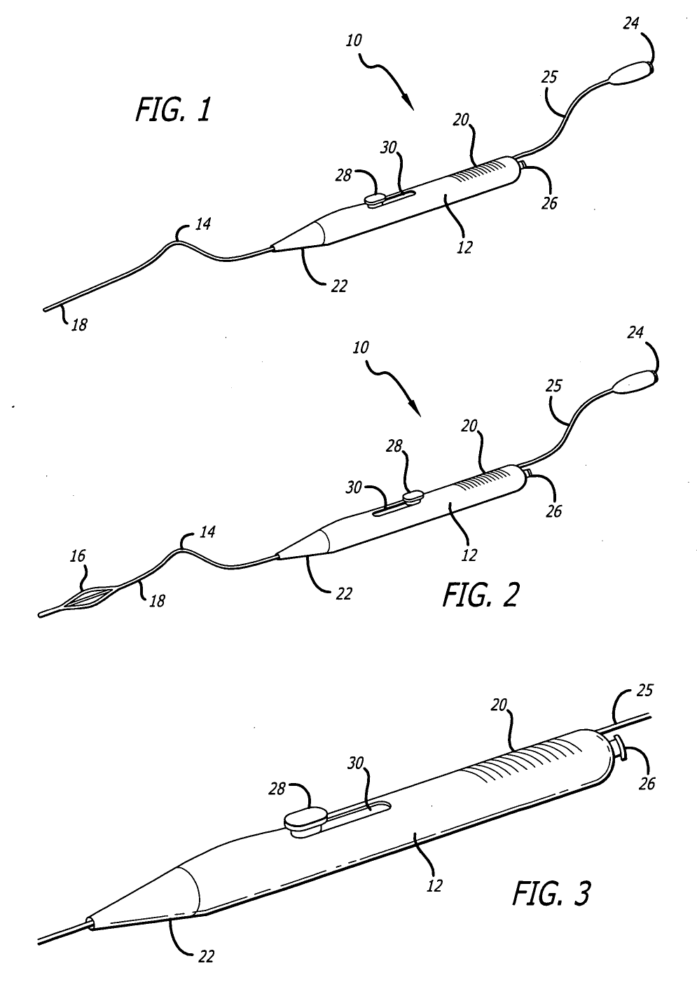

FIGS. 1 and 2 show an embodiment of a flexible thermography catheter. As shown, the thermography catheter 10 has a handle 12 coupled to or otherwise in communication with an elongated body 14. An expandable body 16 is positioned on or proximate to the distal section 18 of the elongated body 14. FIG. 1 shows the catheter 10 wherein in the expandable body 16 of the thermography catheter 10 is in a constrained non-deployed state. FIG. 2 shows the expandable body 16 in an expanded deployed state. As shown in FIGS. 1-3, the handle 12 may include a handle body 20 having an elongated body receiver 22 capable of receiving the elongated body 14 therein. In the illustrated embodiment the elongated body receiver 22 is detachably coupled to the handle body 20 thereby permitting the elongated body 14 to be replaceable. In an alternate embodiment, the elongated body receiver 22 may be integral to the handle body 20. In addition, the handle body 20 may include a sensor coupler 24 attached to a sensor cable 25, thereby permitting the thermography catheter 10 to coupled to any number of analyzing devices, such as a data display module (not shown).

Exemplary data display modules are disclosed in U.S. Patent Application Serial No. 60/379,437, entitled “Method and System for Treating Vulnerable Vascular Plaque,” filed on May 7, 2002, and U.S. Patent Application Serial No. 60/431,329, entitled “CATHETER CALIBRATION SYSTEM AND METHOD,” filed on Dec. 6, 2002, both of which are incorporated herein by reference in their entirety herein. Other analyzing devices which may be used with the thermography catheter 10 can include computers, display devices, including data display modules, amp meters, ohm meters, pH analyzers, electromagnetic analyzers, temperature analyzers, and blood analyzers.

In one embodiment, the handle body 20 may include a proximal port 26 capable of receiving at least one guidewire (not shown) therein. In an alternate embodiment, the proximal port 26 may be configured to receive fluid therethrough, thereby permitting the user to irrigate or flush the elongated body 14, the section 18, and/or the expandable body 16. For example, the proximal port 26 may include a luer-type connector capable of sealably engaging an irrigation device such as a syringe. An elongated body actuator 28 may be positioned within an actuator recess 30 formed on the handle body 20. The thermography catheter 10, or the various components thereof, may be manufactured from a variety of materials, including, for example, plastics, polytetrafluoroethylene (PTFE), polyether block amide (PEBAX), thermoplastic, polyimide, silicone, elastomer, metals, such as stainless steel, titanium, shape-memory alloys such as Nitinol, or other biologically compatible materials. In addition, the thermography catheter 10 may be manufactured in a variety of lengths, diameters, dimensions, and shapes. For example, in one embodiment the elongated body 14 may be manufactured to have length of about 130 cm to about 200 cm. In another embodiment, the elongated body 14 of the thermography catheter 10 may be manufactured to have a transverse dimension of about 0.02 inch to about 0.06 inch, thereby permitting the thermography catheter 10 to be configured for insertion into the coronary vasculature of a patient.

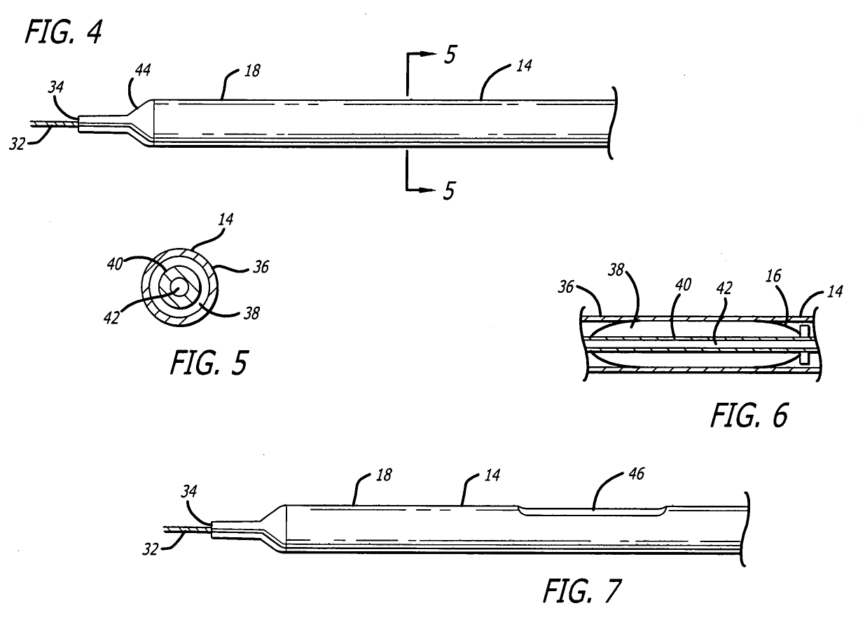

FIGS. 4-6 illustrate the elongated body 14 of the thermography catheter 10 in a non-deployed state. FIG. 4 shows the elongated body 14 prior to deployment of the expandable body 16 (see FIG. 2) from the distal section 18. The distal tip 44 of the distal section 18 may include at least one guidewire port 34 capable of receiving a guidewire 32 therein. As shown in FIGS. 5 and 6, the elongated body 14 may include an outer sleeve 36 forming a sleeve lumen 38 and housing an inner body 40 therein. In one embodiment, the outer sleeve 36 may be manufactured from a material, such as PEBAX, having a wall thickness of about 0.0005 inch to about 0.003 inch. In another embodiment, the outer sleeve 36 has a wall thickness of about 0.0015 inch to about 0.0025 inch. In a non-deployed state, the expandable body 16 of the thermography catheter 10 may be positioned within the sleeve lumen 38 formed by the outer sleeve 36. FIG. 6 shows the expandable body 16 positioned within the sleeve lumen 38 formed within the outer sleeve 36 prior to deployment. As shown, the expandable body 16 may be compressed inwardly by an inner surface of the outer sleeve 36 and located within the sleeve lumen 38. In an alternate embodiment, the elongated body 14 may be manufactured without an outer sleeve. The inner body 40 defines an internal passage 42 therein. In the illustrated embodiment, an internal passage 42 is formed within the inner body 40, however, the internal passage may not be present in some embodiments. In another embodiment, the inner body 40 may define a plurality of internal passages therein. The internal passage 42 formed in the inner body 40 may be in communication with the guidewire port 34 located on the distal tip 44 and may be capable of receiving the guidewire 32 therein (see FIG. 4).

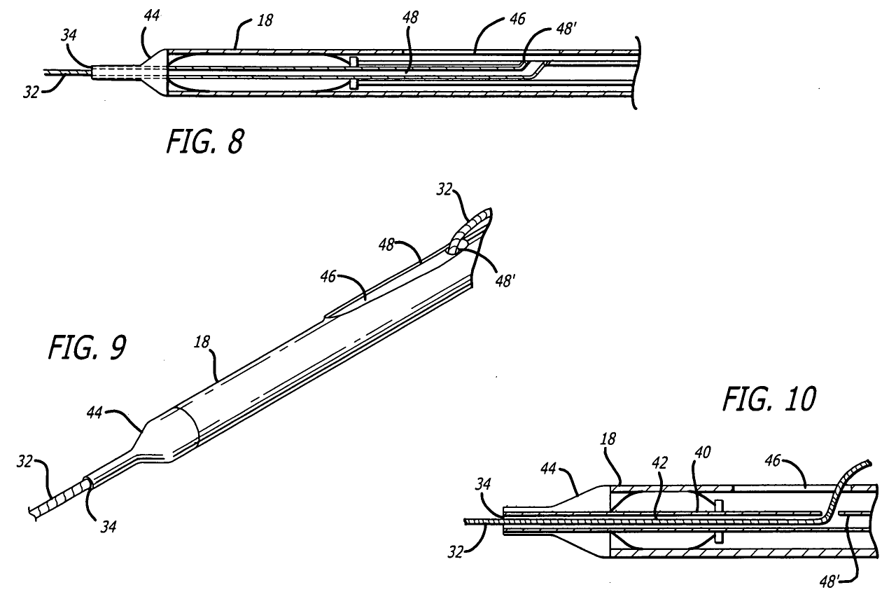

FIGS. 7-10 illustrate alternate embodiments of the elongated body 14. Commonly assigned U.S. patent application Ser. No. 10/253,391, entitled “Thermography Catheters Allowing for Rapid Exchange and Methods of Use,” filed on Sep. 23, 2002, which is incorporated by reference in its entirety herein, describes thermography catheters allowing for rapid exchange of a catheter over a guidewire. As shown in FIG. 7, the elongated body 14 may include a guidewire exit slot 46 thereon. As shown in FIG. 8, a guidewire lumen 48 may be secured to the guidewire port 34 on the distal tip 44. A proximal end of the guidewire lumen 48 communicates with a guidewire exit port 48′ in the inner body 40, thereby permitting the guidewire port 34 to communicate with the guidewire exit slot 46. The guidewire lumen 48 may be secured to the guidewire port 34 using, for example, adhesives or bonding agents, mechanical couplers, pins, snap-fit devices, and other coupling devices known in the art. As a shown in FIG. 9, the guidewire 32 may be introduced into the guidewire port 34 and made to traverse the guidewire lumen 48, exiting the thermography catheter 10 through the guidewire exit port 48′ positioned in the guidewire exit slot 46. The guidewire exit slot 46 may be formed at a variety of distances along the elongated body 14. In one illustrated embodiment the distance between the guidewire port 34 and the guidewire exit slot 46 is about 5 cm to about 40 cm.

FIG. 10 shows an alternate embodiment of the thermography catheter 10 wherein the internal passage 42 formed within the inner body 40, communicates with the guidewire port 34 and the guidewire exit slot 46 via a guidewire exit port 48′ in the inner body 40. As a result, the guidewire 32 enters the distal section 18 of the thermography catheter 10 through the guidewire port 34, traverses the internal passage 42 of the inner body 40, and exits the elongated body 14 through the guidewire exit port 48′ and the guidewire exit slot 46.

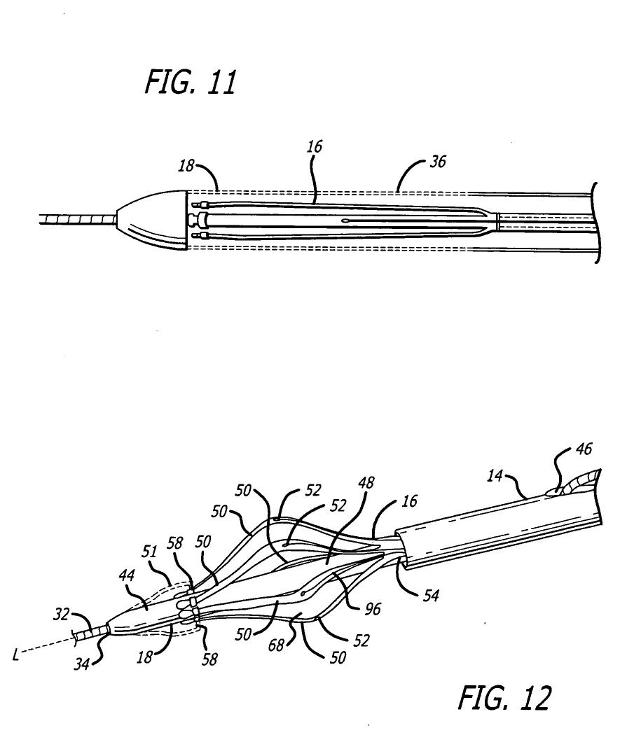

FIG. 11 shows the expandable body 16 prior to deployment. As shown, the expandable body 16 is positioned proximate to the distal section 18 of the thermography catheter 10 and compressed inwardly by an inner surface of the outer sleeve 36. The outer sleeve 36 may be in communication with or attached to the elongated body actuator 28 positioned within the actuator recess 30 located on the handle body 20 of the handle 12 (see. FIG. 3). The rearward movement of the elongated body actuator 28 within the actuator recess 30 results in the outer sleeve 36 retracting rearwardly from the distal tip 44 (see FIG. 4), thereby permitting the expandable body 16 to expand radially. In an alternate embodiment, the outer sleeve 36 may remain stationary while the inner body 40 may be capable of moving in telescopic relation thereto. For example, the inner body 40 may communicate with the elongated body actuator 28 positioned within the actuator recess 30 located on the handle body 20 of the handle 12 (see. FIG. 3). The forward movement of the elongated body actuator 28 within the actuator recess 30 results in the inner body 40 extending distally from the handle 12 (see FIG. 1), thereby advancing the expandable body 16 beyond the outer sleeve 36 and permitting the expandable body 16 to expand radially.

FIG. 12 shows the expandable body 16 in a deployed state wherein at least one support arm 50 has expanded outwardly. In the illustrated embodiment, the expandable body 16 includes five support arms 50. At least one sensor 52 may be positioned on at least one of the support arms 50. The support arms 50 may be manufactured from a variety of materials, including, for example, supereleastic or shape memory alloys such as Nitinol, and other metals such as titanium, elgiloy® and stainless steel. The support arms 50 could also be made of polymers or polymer. composites that include, thermoplastics, resins, carbon fiber, and like materials. In the illustrated embodiment, the support arms 50 are secured to a deployment support member 54 which may be secured to the inner body 40 (see FIG. 5) in a variety of ways, including, adhesively bonded, laser welded, in mechanical coupled, or integrally formed. In an alternate embodiment, the support arms 50 may be secured to the inner body 40 directly, thereby eliminating the need for a deployment support member 54.

In one embodiment, the support arms 50 may be coupled to the deployment support member 54 using an adhesive, such as Loctite 3311 adhesive or any other biologically compatible adhesive. In an alternate embodiment, the expandable body 16 may be manufactured by laser cutting or forming the at least one support arm 50 from a substrate. For example, any number support arms 50 may be laser cut within a Nitinol tube or cylinder, thereby providing a slotted expandable body. Thereafter, the one or more support arms 50 may be formed in a deployed state as shown in FIG. 12 wherein at least one support arm 50 is flared outwardly from the longitudinal axis L of the expandable body 16. In the illustrated embodiment, the guidewire lumen 48, capable of receiving the guidewire 32 therein, longitudinally traverses the expandable body 16. The guidewire lumen 48 is in communication with the guidewire port 34 on the distal section 18 and guidewire exit slot 46 located on the elongated body 14. In an alternate embodiment, the guidewire lumen 48 may be in communication with the guidewire port 34 on the distal section 18 and the proximal port 26 located on the handle body 20 (see FIGS. 1 and 2). In the illustrated embodiment, a retainer sleeve 51 may be positioned over a section of the support arms 50 to provide a transition between the distal section 18 and the support arms 50.

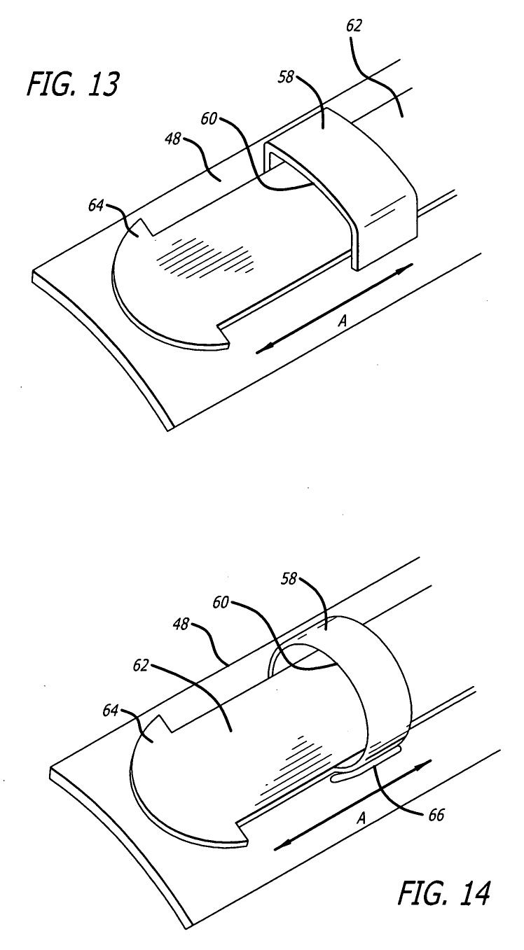

FIGS. 13 and 14 illustrate two embodiments of a support arm retainer 58 which may be used to secure a support arm 50 to the guidewire lumen 48. Alternatively, the support arm retainers 58 may be positioned on the deployment support member 54, the inner body 40, or the distal tip 44. The support arm retainer 58 creates a support arm channel 60 capable of movably receiving the support arm body 62 therethrough (as shown by arrow A), while preventing the passage of the retaining device 64 therethough. In the embodiment shown, the support arm retainer 58 allows for axial translation of the portion of the support arm 50 captured by the support arm retainer 58, but does not allow substantial translation in a radial direction of the captured portion of the support arm 50.

In the embodiment illustrated in FIG. 12, each support arm 50 is coupled to the guidewire lumen 48 using a support arm retainer 58, thereby permitting each support arm 50 to move independently relative to the guidewire lumen 48 and the other support arms 50. The ability of the support arms 50 to independently move within the support arm retainer 58 results in the creation of an expandable body 16 offering flexibility, while permitting the support arms 50 to remain in contact with a vessel wall (not shown) when traversing a tortuous pathway. More particularly, when the expandable body 16 is in a non-deployed state, the ability of the support arms 50 to move independently of each other in an axial direction reduces the shear resistance and results in a more flexible catheter than a catheter wherein the axial movement is coupled or otherwise restricted. In addition, when the expandable body 16 is in a deployed state, the ability of the support arms 50 to move independently facilitates contact of each of the support arms 50 with the vessel wall without applying excessive force thereto, thereby decreasing or eliminating the likelihood of injury to the vessel. Maximizing contact of each of the support arms 50 with the vessel wall in turn maximizes contact of the sensors 52 with the vessel wall which can be important in some embodiments for obtaining accurate sensor readings.

As shown in FIG. 13, the support arm retainer 58 may be rectangular in cross section. In an alternate embodiment, FIG. 14 shows a circular support arm retainer 58 coupled to the guidewire lumen 48 while permitting the independent movement (illustrated by arrow A) of the support arm 50 therethrough. The support arm retainer 58 may be manufactured in a variety of shapes and configurations. The support arm retainer 58 shown in FIG. 14 has a tubular configuration with a circular transverse cross sections, but could also have an elliptical, square, rectangular or triangular cross sectional configuration. Non-circular cross sectional configurations for the retainers 58 can be used to prevent twisting or torquing at the arms 50 within the retainers 58 while still permitting relative longitudinal movement there between.

In another embodiment, the support arm retainer 58 may be formed from a ring of material, for example, a polyester heat shrinkable tubing and may include a number of openings capable of receiving at least one support arm 50 therethrough. The support arm retainer 58 may be manufactured from a variety of materials, including, without limitation, shrink-fit materials, polyesters, thermoplastics, plastics, metals such as titanium, stainless steel titanium, shape memory alloys such as Nitinol, elastomer, or other materials capable of receiving and constraining the at least one support arm 50 therein. The support arm retainer 58 may be secured to the guidewire lumen 48, the deployment support member 54, or the distal tip 44 (see FIGS. 6 and 12), in a variety of ways, including, for example, laser welded, adhesively bonded, or integrally formed. FIG. 14 shows the support arm retainer 58 adhesively bonded at a bond point 66 to the guidewire lumen 48 using a biologically compatible adhesive, such as Loctite 3311 adhesive.

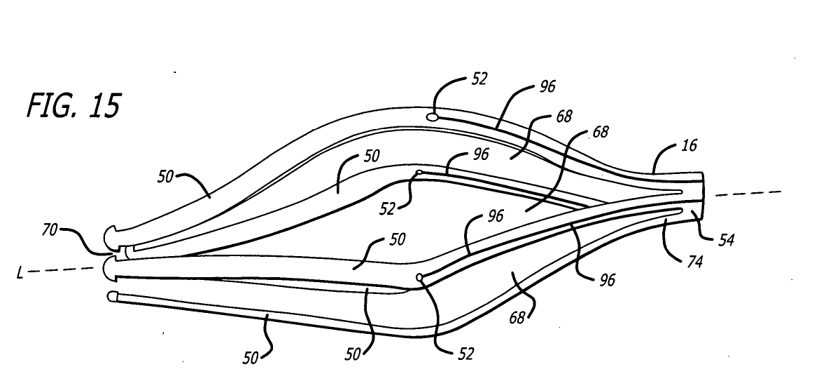

Referring to FIG. 15, the expandable body 16 may include any number of support arms 50 separated by one or more slots 68. The expandable body 16 may be generally hollow in design and may define an expandable body passage 70 capable of receiving the guidewire 32 or the guidewire lumen 48 therethrough (see FIG. 12). In the illustrated embodiment, the support arms 50 are symmetrically positioned around the expandable body passage 70. In an alternate embodiment, the support arms 50 are asymmetrically positioned around the expandable body passage 70. The expandable body 16 may be manufactured from a variety of materials, including, for example, shape memory alloys such as Nitinol, metals such as stainless steel and titanium, polymers, composite materials, and like materials. In one embodiment, the expandable body 16 may be formed from a Nitinol hypodermic tube having at least one slot 68 formed therein, thereby defining at least one support arm 50 thereon.

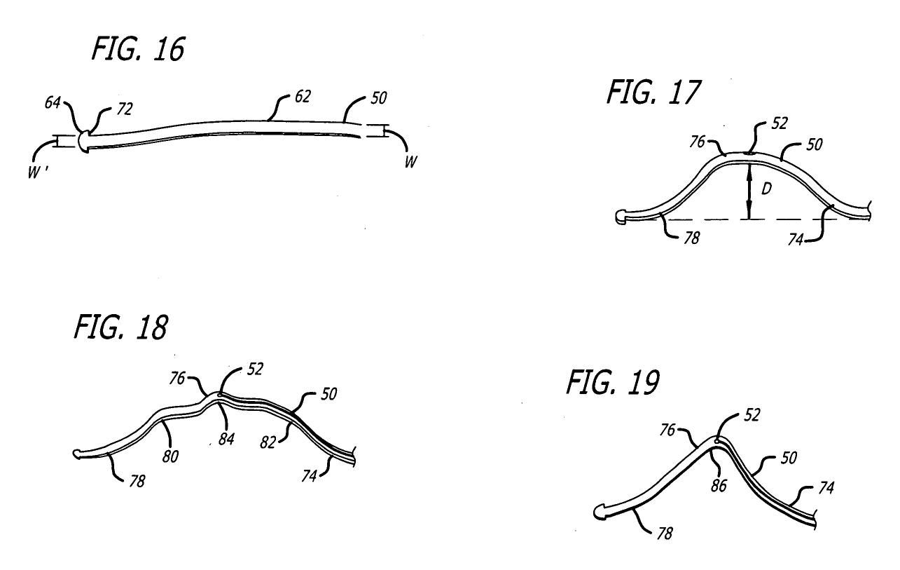

Referring again to FIG. 15, the expandable body 16 is shown removed from the thermography catheter 10 (see FIG. 2). As shown in FIG. 16, the support arms 50 may include at least one retaining device 64 positioned thereon. In the illustrated embodiment, the retaining device 64 includes a retaining flange 72 having a width W′ which is greater than the width W of the support arm body 62 and the support arm channel 60 (see FIG. 13), thereby preventing the retaining device 64 from traversing the support arm channel 60 of the support arm retainer 58 (see FIG. 13). Alternate embodiments of the retaining device 64 are contemplated, and include, without limitation, buttons, tabs, pins, spots, or other devices deposited on, attached to, or integral with the at least one support arm 50 which are capable of preventing the retaining device 64 from traversing the support arm channel 60 of the support arm retainer 58 (see FIG. 13).

During manufacture, the at least one support arm 50 is formed to assume a deployed position in a relaxed state as shown in FIG. 17, wherein the support arm 50 are flared outwardly a distance D from the longitudinal axis L of the expandable body 16. The application of force to the apex of the actuate support arm 50 decreases the curvature of the support arm 50 resulting in a corresponding decrease in the distance D.

As shown in FIGS. 16-19, the support arms 50 include a proximal-section 74 capable of coupling to the deployment support member 54 as shown in FIG. 15. Alternatively, the proximal section 74 may be capable of coupling to the elongated body 14 or the inner body 40. The support arms 50 may also include a medial section 76 configured to be positioned proximate to or in contact with the vessel wall when the expandable body 16 is deployed. In addition, the support arms 50 include a distal section 78 having a retaining device 64 coupled thereto (see FIG. 16). At least one sensor 52 may be positioned on the medial section 76 of the at least one support arm 50 thereby enabling the at least one sensor 52 to contact the vessel wall when the expandable body 16 is deployed (see FIG. 12). As illustrated in FIG. 17, the general shape of the at least one support arm 50 substantially ensures that only the medial section 76 of the at least one support arm 50 contacts the vessel wall when the expandable body 16 is deployed (see FIG. 12). The at least one support arm 50 may be manufactured in any variety of shapes having a medial section 76 configured to be positioned proximate to or in contact with the vessel wall including arcuate shape, bell shapes, and smooth transitional shapes. For example, FIGS. 18 and 19 show alternate embodiments of the at least one support arm 50. As shown in FIG. 18, the at least one support arm 50 may include a smoothed-step configuration having at least a transition located between at least one of the distal or proximal section 78, 74, respectively, and the medial section 76. In the illustrated embodiment, a first transition 80 is positioned between the distal section 78 and the medial section 76. A second transition 82 is located between the proximal section 74 and the medial section 76. A sensor protrusion 84 having a sensor 52 located thereon is positioned on the medial section 76 thereby enabling the sensor 52 to contact the vesselwall. FIG. 19 shows another embodiment of the at least one support arm 50 having an abruptly curved distal section 78 coupled to an abruptly curved proximal section 74 with a sensor peak 86 configured to receive sensor 52 located at the medial section 76.

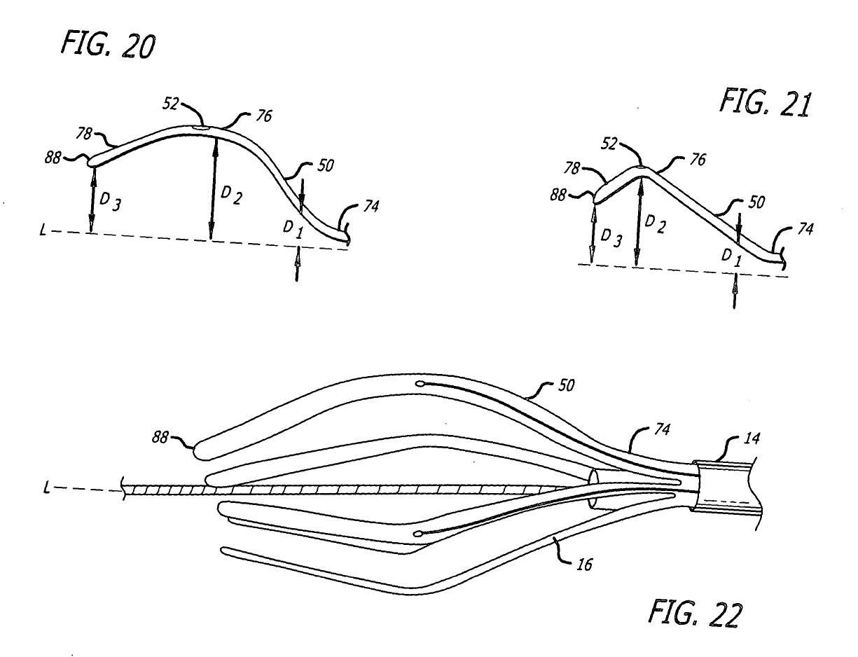

FIGS. 20-22 show an alternate embodiment of the expandable body 16. As shown in FIGS. 20-22, the expandable body 16 may include at least one support arm 50 secured to the elongated body 14 at the proximal section 74. The medial section 76 of the at support arm 50 may include at least one sensor 52 located thereon. The distal section 78 of the at least one support arm 50 includes atraumatic tip 88. In the illustrated embodiment, the atraumatic tip 88 may comprise a rounded, tapered, or blunted end. The atraumatic tip 88 may be integral to the at least one support arm 50, or, in the alternative, may coupled to the at least one support arm 50. For example, the atraumatic tip 88 may comprise a silicone device adhesively coupled to at least one support arm 50. As shown in FIGS. 20-22, the proximal section 74 of the at least one support arm 50 is positioned a distance D1 from the longitudinal axis L of the expandable body 16. The medial section 76 of the at least one support arm 50 is positioned a distance D2 from the longitudinal axis L of the expandable body 16, while the distal section 78 of the at least one support arm 50 is positioned a distance D3 from the longitudinal axis L of the expandable body 16. As illustrated in FIGS. 20-22, when the expandable body 16 is deployed the medial section 76 of the at least one support arm 50 has the greatest radial displacement from the longitudinal axis L of the expandable body 16 such that distances D1 and D3 are less than distance D2. As a result, the at least one sensor 50 positioned on the medial section 76 of the at least one of the support arm 50 is permitted to contact the vessel wall.

Referring again to FIG. 12, the ability of support arms 50 to move independently with respect to the guidewire lumen 48 and the other support arms 50 results in the formation of a flexible expandable body 16 capable of traversing tortuous pathways. The support arms 50 of the expandable body may be manufactured in a variety of shapes, lengths, widths, and thickness to promote the flexibility of the individual support arms 50. For example, in one embodiment the support arms 50 may have a length of about 0.2 inch to about 2.0 inch, more specifically, a length of about 0.4 inch to about 1.0 inch. Similarly, the support arms 50 may be manufactured from a material having a thickness of about 0.0015 inch to about 0.007 inch. More specifically, in one embodiment, the support arms 50 have a thickness of about 0.0025 inch to about 0.0045 inch.

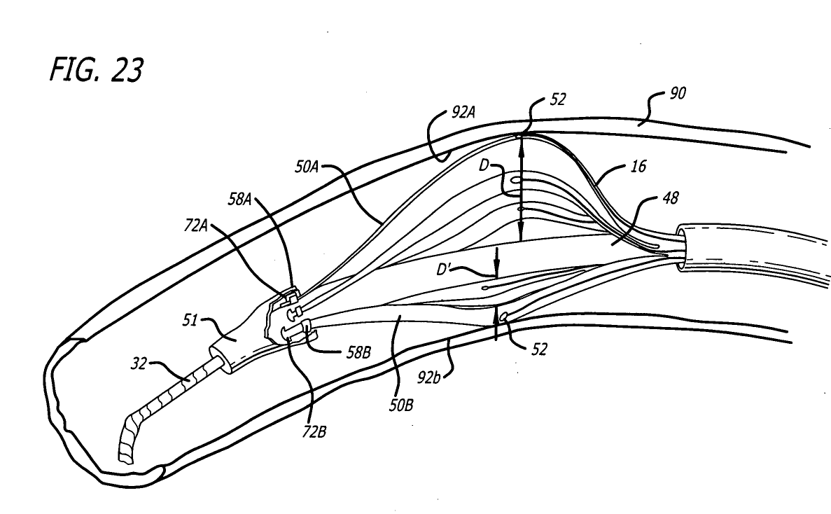

FIG. 23 shows the expandable body 16 deployed within an arcuate blood vessel 90. As shown, an apex of a first support arm 50A is extended a first distance D from the guidewire lumen 48 while permitting a sensor 52 positioned thereon to remain in contact with the vessel wall 92A. The first retaining flange 72A is positioned proximate to the first support arm retainer 58A within the retainer sleeve 51. A second support arm 50B has an apex that is positioned a second distance D′ from to the guidewire lumen 48 while permitting a sensor 52 positioned thereon to remain in contact with the vessel wall 92B, wherein the second distance D′ is smaller than the first distance D. The second retaining flange 72B is positioned distally from the second support arm retainer 58B within the retainer sleeve 51. As a result, sensors 52 positioned on each of the support arms 50A, 50B remain in contact with the vessel wall 90 despite the disparity between distances D and D′.

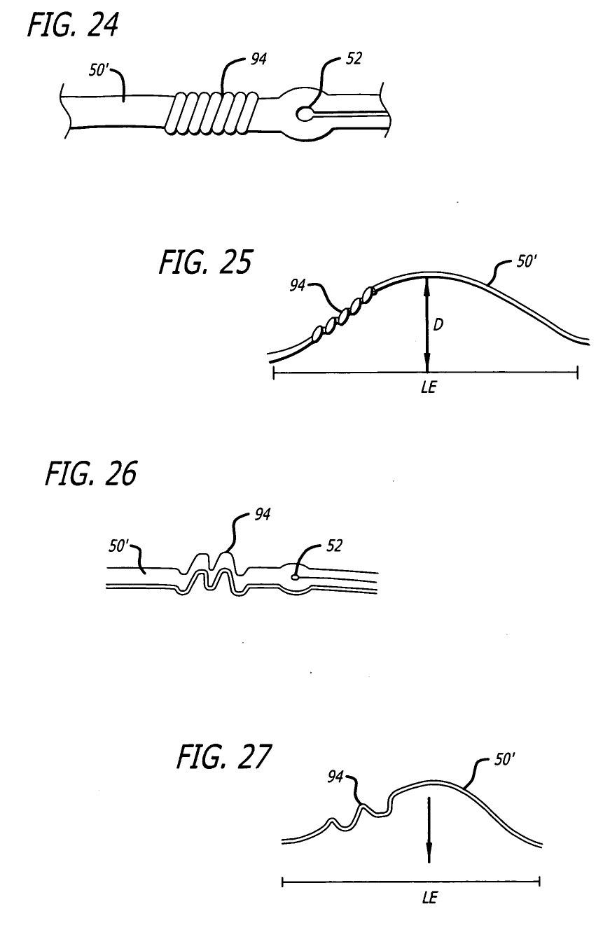

FIGS. 24-27 show alternate embodiments of one support arm 50′ having a flex member 94 coupled to or formed thereon. In the illustrated embodiments, the flex member 94 is positioned distal to the sensor 52, however, the flex member 94 may be positioned anywhere along the body of the support arm 50′. Similar to the embodiment illustrated in FIG. 17, the application of force to the apex of the arcutate support arm 50′ results in a decrease of the curvature of the support arm 50′ and a corresponding decrease in the distance D. In addition to enhancing radial flexibility, the flex member 94 may permit the support arm 50′ to flex longitudinally along the line LE, thereby permitting the length of the support arm 50′ to vary. Such an embodiment would facilitate independent radial movement of the support arm 50′ even if both the proximal and distal end of the support arm 50′ are fixed with regard to relative longitudial movement.

Referring again to FIG. 15, each of the support arms 50 includes at least one sensor 52 thereon. Exemplary sensors 52 include, without limitation, ultrasonic sensors, flow sensors, thermal sensors, such as thermocouples, thermistors and infrared sensors, electrical contact sensors, conductivity sensors, electromagnetic detectors, chemical sensors, and pH sensors. For example, in one embodiment, a thermocouple constructed of a first thermocouple conductor and second thermocouple conductor may be used as a sensor 52. The first thermocouple conductor may be constructed of chromel and second thermocouple conductor may be constructed of constantan. The junction of the chromel and constantan materials, which forms the sensor 52, yields the desired thermocouple properties that allow accurate temperature measurement. Other materials suitable for yielding the desired thermocouple properties could also be used for the first and second thermocouple conductors. In one embodiment, the thermocouple may be capable of detecting thermal discontinuities or variations in vessel wall temperature, thereby providing a thermography catheter capable of locating inflamed or vulnerable plaque on the wall of a blood vessel in vivo.

In an alternate embodiment, at least one sensor 52 may be comprised of flexible circuits integrated into at least one support arm 50. A particular flexible circuit that is applicable to the thermography catheter is disclosed in commonly assigned U.S. patent application Ser. No. 09/938963, filed Aug. 24, 2001, which is incorporated by reference in its entirety herein.

The flexible circuit is comprised of polymer thick film flex circuit that incorporates a specially formulated conductive or resistive ink that is screen printed onto the flexible substrate to create the thermal sensor circuit patterns. This substrate is then adhered to the surface of each of the support arms 50. In an alternate embodiment, the substrate can be adhered to independently expandable, resilient body arms which are not part of an expandable body 16. As with all of the embodiments, the catheter can be provided with the appropriate number of body arms, such as four, five, six, or more.

As a result, the thermography catheter 10 may be capable of simultaneously examining a number of characteristics of tissue within the body of a patient, including, for example, vessel wall temperature, blood temperature, fluorescence, luminescence, flow rate, and flow pressure. As shown in FIG. 15, the sensor 52 may be positioned on or near the apex of the arcuate support arms 50 when the expandable slotted body 16 is deployed in an expanded state, thereby permitting the sensors 52 to contact a vessel wall. In an alternate embodiment, the at least one sensor 52 may be positioned on the support arms 50 at any radial distance less than the radial distance of the apex of the arcuate support arms 50 relative the longitudinal axis L of the expandable body 16 when the expandable body 16 is in a deployed state, thereby preventing the at least one sensor 52 from contacting a vessel wall when the expandable body 16 is deployed to an expanded state. At least one sensor conduit 96 may be in communication with at least one the sensor 52 positioned on or proximate to the expandable body 16 (See FIG. 12). The sensor conduit 96 may be in communication with the sensor cable 25 on the handle 12 (see FIGS. 1 and 2). The at least one sensor conduit 96 may traverse the elongated body 14 through the sleeve lumen 38, the internal passage 42 (see FIG. 6), or both. The at least one sensor 52, the sensor conduit 96, or both may be secured to the support arm 50 in a variety of ways, including, for example, adhesively and mechanically. For example, in one embodiment, a sensor 52 may be adhesively bonded to the support arm 50 using Loctite 3311 or any other biologically compatible adhesive.

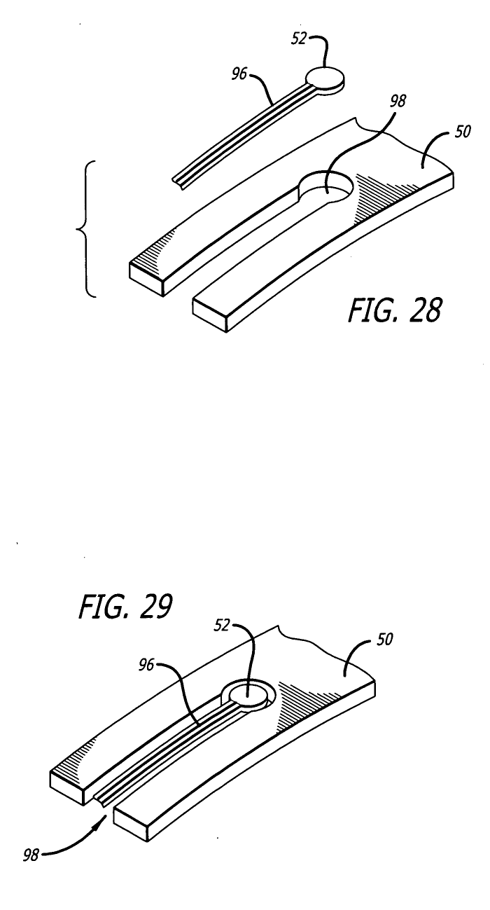

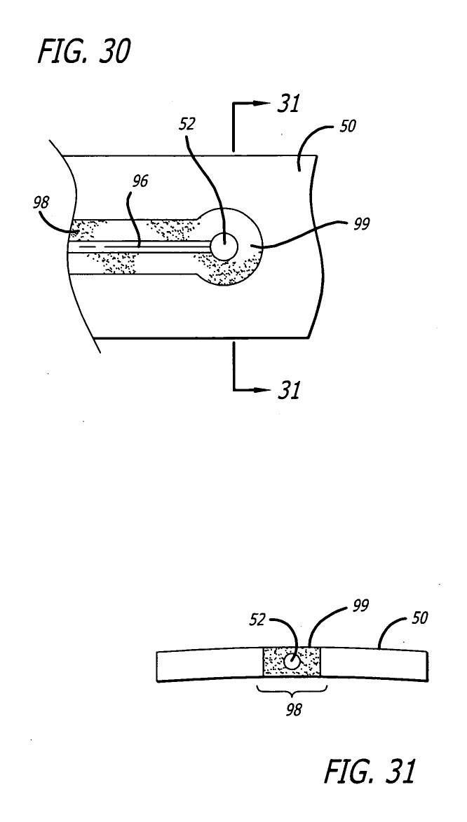

FIGS. 28-31 show alternate embodiments of a support arm 50 of the thermography catheter 10 having a sensor slot 98 formed therein. As shown in FIGS. 28 and 29, the sensor slot 98 formed on the support arm 50 may be capable of receiving a sensor 52, a sensor conduit 96, or both therein. As shown in FIGS. 30 and 31, the sensor 52 may be secured to the support arm 50 within the sensor slot 98 with an epoxy or other biological compatible adhesive material 99, thereby reducing the profile of the expandable slotted body 16 when compared to prior art devices. An example of such a device is disclosed in U.S. patent application Ser. No. 10/099,409, filed Mar. 15, 2002, which is incorporated by reference in its entirety herein. As a result, the thermography catheter 10 may be effectively used in smaller diameter locations within the body as compared with prior art systems. The sensor slot 98 may be formed in the support arm 50 by laser machining or chemically etching the outer surface of an expandable tube or sheet, prior to forming each of the individual support arms 50 that make up the expandable body 16.

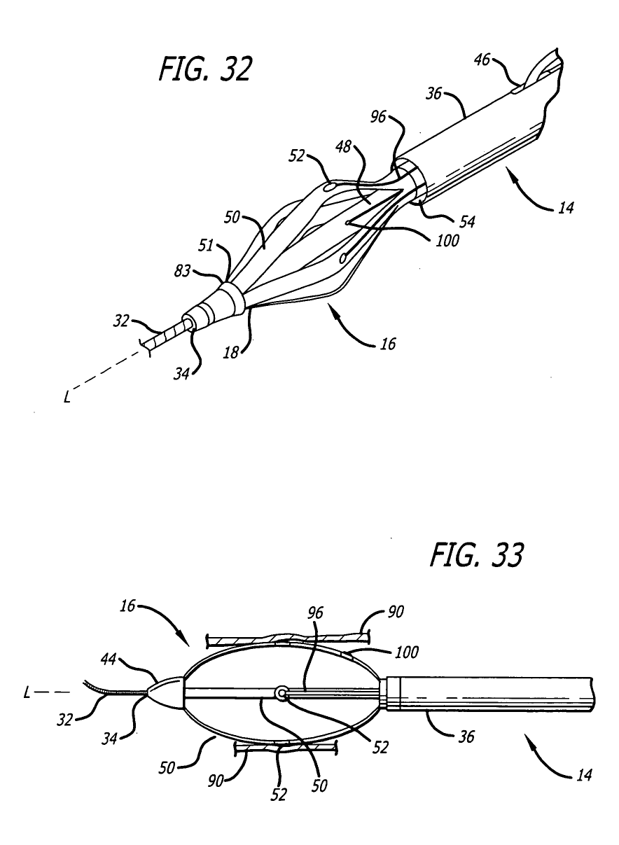

FIGS. 32 and 33 show an embodiment of the expandable body 16 of the thermography catheter 10 during use. As shown, the support arms 50 have expanded outwardly from the longitudinal axis L of the expandable body 16, thereby permitting the sensors 52 located on the support arms 50 to contact the internal surface of the vessel wall 90 to be examined. As shown, the retainer sleeve 51 is positioned over the support arm retainers 58, the retaining devices 64, or both, (see FIGS. 13 and 14), thereby preventing the support arm retainers 58 from contacting the vessel wall 90 (see FIG. 12) and causing trauma to the vessel wall 90, damaging the support arm retainers 58, or both. The sensor conduits 96 couple the sensors 52 to the sensor coupler 24 (see FIGS. 1 and 2) through the elongated body 14. The expandable body 16 may include at least one ancillary sensor 100 thereon. As shown in FIG. 32, the ancillary sensor 100 may be positioned on the guidewire lumen 48. In the alternative, as shown in FIG. 33, at least one ancillary sensor 100 may be positioned on at least one support arm 50. Exemplary ancillary sensors 100 include, without limitation, ultrasonic sensors, flow sensors, thermal sensors, blood temperature sensors, electrical contact sensors, conductivity sensors, electromagnetic detectors, chemical sensors, pH sensors, and infrared sensors. For example, in one embodiment the ancillary sensor 100 may comprise a blood sensor positioned on the guidewire lumen 48 as shown in FIG. 32, thereby permitting the sensors 52 located on the support arms 50 to measure the vessel wall temperature while simultaneously the ancillary sensor 100 measures blood temperature within the vessel.

As shown in FIGS. 32 and 33, the guidewire lumen 48 exits through the deployment support member 54 positioned within the outer sleeve 36 and traverses along the longitudinal axis L of the expandable body 16, eventually connecting to the guidewire port 34 formed in the distal tip 18. As a result, the guidewire port 34 is in communication with the guidewire exit slot 46 formed on the elongated body 14. In the illustrated embodiment the five support arms 50 are expanded outwardly thereby forming a “basket” catheter, although the thermography catheter may include any number of support arms 50.

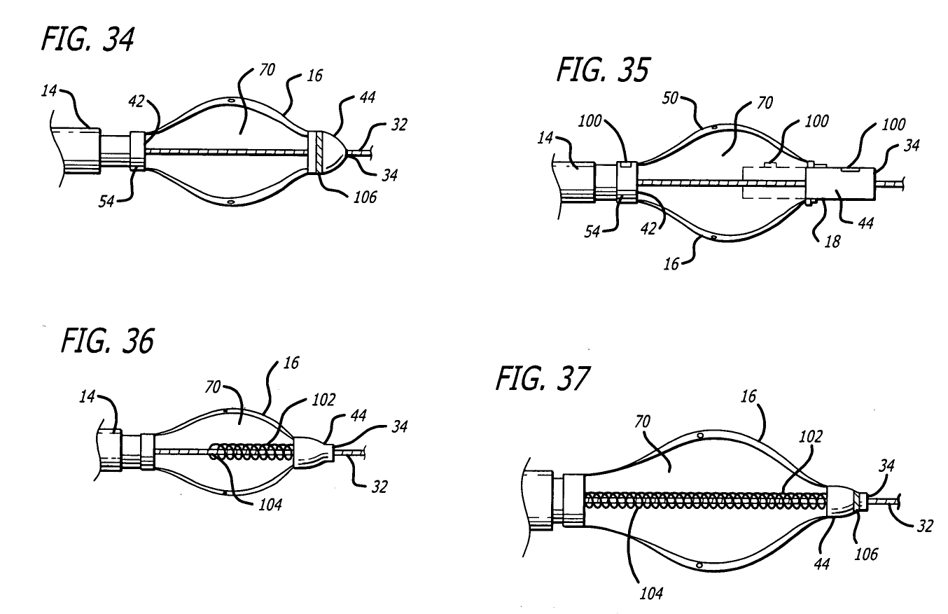

FIGS. 34-37 show several alternate embodiments of the expandable body 16 that may be used in order to improve the flexibility of the expandable body 16. As shown in FIG. 34, the expandable body 16 may be constructed without a guidewire lumen 48 (see FIG. 32) traversing the expandable body 16. The flexible distal tip 44 receives the guidewire 32 through the guidewire port 34. Thereafter, the guidewire 32 traverses the expandable body passage 70, entering the elongated body 14 through the deployment support member 54. FIG. 35 shows a thermography catheter having a section of an elongated distal tip 44. The section of the elongated distal tip 44 extends proximally into the expandable body passage 70 and provides a more rigid distal section 18 of the expandable body 16 as compared to the embodiment shown in FIG. 34, while still providing a flexible thermography catheter 10. As with any of the preceding embodiments, at least one ancillary sensor 100 may be positioned on any one of the support arms 50, the distal tip 44, the deployment support member 54, the expandable body 16, and/or the elongated body 14. Like the previous embodiment, the guidewire 32 traverses the expandable body passage 70, entering the elongated body 16 through the deployment support member 54.

As shown in FIGS. 36 and 37, a coiled member 102 defining a guidewire lumen or a coil passageway 104 located within the expandable body 16 which may be in communication with the guidewire port 34 located on the flexible distal tip 44. The coil passageway 104 may be capable of receiving a guidewire 32 therein. As a result, the coiled member 102 functions as a flexible guidewire passageway. As shown in FIG. 36, the coiled member 102 may be of consistent pitch, tension, and/or transverse dimension. In the alternative, the coiled member 102 may vary in pitch, tension, and/or transverse dimension thereby resulting in an extendable body 16 having a variable flexibility. As shown in FIG. 36, the coiled member 102 defining a coil passageway 104 may partially traverse the expandable body 16. In the alternative, the coiled member 102 defining a coil passageway 104 may be in communication with the elongated body 14 and the distal tip 44, thereby traversing the expandable body passage 70.

FIG. 37 shows one embodiment of an extendable body 16 having a coiled member 102 which varies in pitch and transverse dimension. As shown in FIGS. 34 and 37, the flexible distal tip 44 of the expandable body 16 may include at least one positioning aid 106 thereon. The at least one positioning aid 106 may be positioned on at least one of the flexible distal tip 44, distal section 44, expandable body 16, guidewire lumen 48 (see FIG. 11), deployment support member 54, and/or elongated body 14 (see FIGS. 6 and 12). The positioning aid 106 may include radio-opaque markers, echogenic materials, IVUS devices, magnets or magnetic imaging devices, or other positioning or imaging aides known in the art, and may be incorporated into any of the previous embodiments.

During use, a guidewire 32 (see FIGS. 23 and 32) may be introduced into the blood vessel of a patient using standard percutaneous techniques. Once the guidewire 32 is positioned within the blood vessel, the thermography catheter 10 (see FIG. 1) is introduced into the blood vessel of a patient over the guidewire 32 and advanced to the area of interest. In the alternative, the thermography catheter 10 may be coupled to the guidewire 32 external to the patient and both the guidewire 32 and the thermography catheter 10 may be introduced into the patient and advanced to an area of interest simultaneously (see FIGS. 23 and 32). The thermography catheter 10 may include IVUS or other imaging devices thereon, thereby permitting the user to precisely position the thermography catheter 10 within the blood vessel. The expandable body 16, which is positioned within the outer sleeve 36 (see FIG. 6) when introduced into the blood vessel, is positioned within the blood vessel proximate to the area of examination. Thereafter, the user operates the elongated body actuator 28 located on the handle 12 to a deployed positioned within the actuator recess 30 (see FIG. 1-3). The rearward operation of the elongated body actuator 28 positioned on the handle 12 (see FIGS. 1 and 2) results in the outer sleeve 36 retracting rearwardly, thereby exposing the expandable body 16 and permitting the expandable body 16 to return to a relaxed, expanded state wherein the one or more support arms 50 flare outwardly (see FIG. 23).

As a result, the at least one sensor 52 located on the at least one support arm 50 contacts the vessel wall thereby enabling the measurement of the vessel wall temperature. Simultaneously, if provided, the ancillary sensor 100 (see FIG. 32) located proximate to the expandable body 16 may measure the blood temperature within the blood vessel without contacting the vessel wall 90 (see FIG. 33). The vessel wall temperature measured with the sensor 52 and blood temperature measurements measured with the ancillary sensor 100 if provided, are sent to a analyzer (not shown) via at least one sensor conduit 96. In the course of the temperature measurement, the distal section 18 of the thermography catheter may be retracted proximally or advanced distally in order to determine a temperature gradient of the vessel wall over a longitudinal length of the vessel. Once the measuring process is completed the user returns the elongated body actuator 28 located on the handle 12 to a non-deployed position within the actuator recess 30 (see FIGS. 1 and 3). As a result, the outer sleeve 36 advances towards the distal section 18 (see FIG. 4). While advancing towards the distal section 18, an inner wall of the outer sleeve 36 engages and compresses the expandable body 16 inwardly, thereby permitting the expandable body 16 to be received within the sleeve lumen 38 and returning the expandable body 16 to a non-deployed state (see FIG. 11). Prior to removing the thermography catheter 10 from the blood vessel, the user may delivery a therapeutic agent to an area of interest with the thermography catheter 10. Thereafter, the thermography catheter 10 and the guidewire 32 may be removed from the patient and the entry incisions may be closed.

During the measurement process, a variety of analyzers (not shown) may be used to display the measured results. The measured results may be illustrated in a variety of ways, including, for example, bar graph, two-dimensional chart, and a three-dimensional image. For example, U.S. Patent Application Serial No. 60/379,437, which is incorporated by reference in its entirety herein, discloses several device for illustrating the measured results.

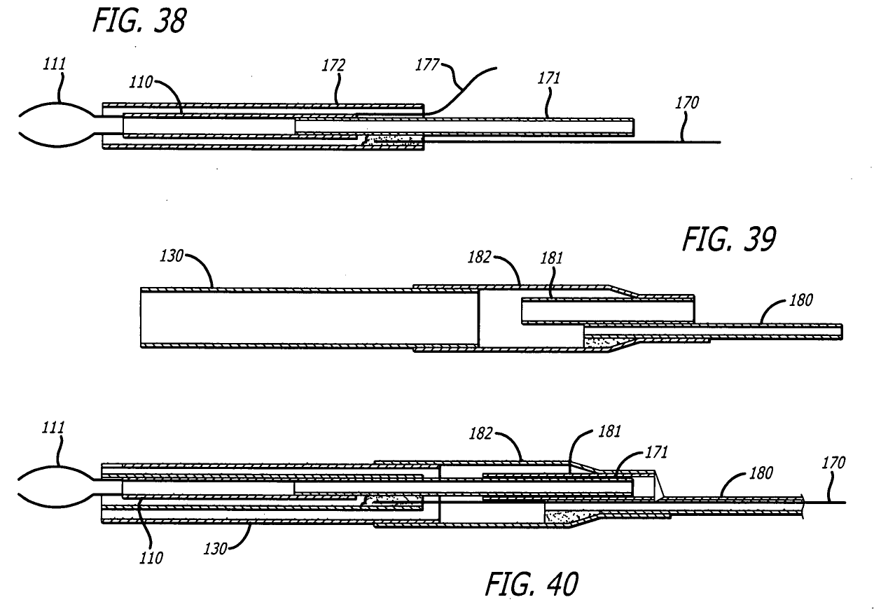

In another embodiment, a thermography catheter having an inner assembly that fits within an outer assembly is shown in FIGS. 38, 39, and 40. The inner assembly comprises elongate member 170, e.g., a mandrel, as shown in FIG. 38. A first tubular member 171 is bonded adjacent the distal end of mandrel 170. Tubular member 171 includes a lumen adapted to slideably receive a guidewire. Expandable member or expansion frame 111, having at least one temperature sensor and being operable between a contracted condition and an expanded condition, is bonded to a distal end of catheter 110. The expandable member or expansion frame 111 shown in FIGS. 38-40, can have a configuration that is the same, or similar to the expandable member 16 or components thereof, discussed above. In addition, any of the expansion frame 111 or expandable members discussed herein can be adapted for use on any of the catheter embodiments, or alternative structures thereof, discussed herein. Second tubular member 172 is disposed about the distal end of catheter 110, but terminates proximal to the expansion frame.

The outer assembly comprises elongate tubular member 180 having a lumen that extends from the proximal end to the distal end of tubular member 180 as shown in FIG. 39. A second tubular member 181 is bonded adjacent the distal end of tubular member 180. Capture sheath 130 is bonded distally to transition tubing 182. Tubular member 181 is shaped to receive tubular member 171 of the inner assembly.

The thermography catheter is assembled by nesting the inner assembly within the outer assembly as shown in FIG. 40. Mandrel 170 is slideably received within tubular member 180 while tubular member 171 is slideably received within tubular member 181. A guidewire is slideably received through the distal end of expansion frame 111 and passes proximally through the lumen of tubular member 171 and tubular member 181. It will be understood that the configuration described above ensures that a clear passage will be maintained at all times for the guidewire to emerge proximally from the guidewire lumen of the inner assembly and the capture sheath. Stated differently, the assembly shown in FIG. 40 will resist rotation of the inner assembly relative to the outer assembly and thereby prevent obstruction of the guidewire passageway.

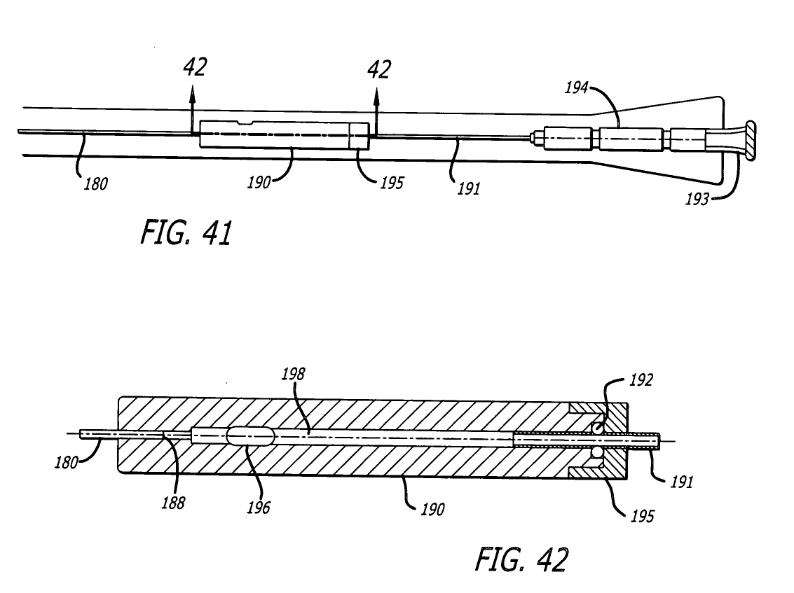

In order to perform flushing the lumen, of tubular member 180 of the outer assembly communicates with a flushing port at the proximal end of the thermography catheter as shown in FIGS. 41 and 42. Tubular member 180 of the outer assembly is bonded proximally to slider body 190, and terminates proximally at flushing port 188. Port 188 communicates with chamber 198 and receives fluid, such as saline, lactated Ringers, or water, for flushing. Chamber 198 is defined byslider body 190 and injection tube 191 to enable relative longitudinal movement without loss of fluid. Slider cap 195 is a further component of the assembly for the dynamic seal. The proximal end of injection tube 191 is bonded to coupling 194, which is connected to luer 193, which provides for input of fluid. A one-way valve, a pressure activated valve, or a luer-activated valve may be included to prevent blood escape when flushing is not needed.

In this manner, fluid injected through luer 193 will pass through coupling 194, injection tube 191, and full chamber 198. Fluid will then pass distally to port 188 and through the lumen of tubular member 180, thereby flushing the annuli between sliding components of the inner and outer assemblies.

What is claimed:

1. A thermography catheter, comprising:
an elongated body having a proximal end, a distal end, and a distal section;
a deployable expandable body disposed on the distal section of the elongated body having at least two support arms configured to be radially displaceable from a longitudinal axis of the expandable body and independently movable in a radial direction in relation to each other when deployed;
the support arms having a proximal section, a distal section, and a medial section, the medial section configured to be radially displaceable a greater distance from the longitudinal axis than the proximal and distal sections; and
a thermal sensor disposed on the medial section of at least one of the support arms and configured to contact the body vessel when deployed.

2. The catheter of claim 1 wherein the elongated body further comprises an inner body coupled to the expandable body.

3. The catheter of claim 2 wherein the inner body defines at least one internal passage therein.

4. The catheter of claim 3 wherein the at least one internal passage comprises a guidewire lumen configured to receive a guidewire.

5. The catheter of claim 2 further comprising an outer sleeve defining a sleeve lumen configured to receive the expandable body therein.

6. The catheter of claim 5 wherein the outer sleeve is configured to controllably retract from the distal section of the elongated body.

7. The catheter of claim 5 wherein at least a portion of the inner body is configured to controllably extend beyond the outer sleeve in telescopic relation thereto.

8. The catheter of claim 4 further comprising a guidewire exit port positioned proximate to the distal section of the elongated body and in communication with the guidewire lumen formed in the at least one internal passage.

9. The catheter of claim 8 wherein the guidewire exit port is positioned about 5 cm to about 40 cm from the distal end of the elongated body.

10. The catheter of claim 1 further comprising a handle secured to the elongated body.

11. The catheter of claim 10 wherein the handle is detachably coupled to the elongated body.

12. The catheter of claim 10 further comprising an elongated body actuator positioned on the handle and located within an actuator recess formed on the handle.

13. The catheter of claim 12 wherein the elongated body actuator is in communication with at least one of an inner body formed within the elongated body and an outer sleeve positioned over the inner body.

14. The catheter of claim 10 further comprising a sensor cable secured to the handle and having a sensor connector secured thereto.

15. The catheter of claim 10 further comprising a proximal port formed on the handle and in communication with the elongated body.

16. The catheter of claim 1 wherein the expandable body is coupled to the distal section of the elongated body.

17. The catheter of claim 1 wherein the expandable body is secured to an inner body positioned within the elongated body.

18. The catheter of claim 1 further comprising a deployment support member configured to couple the expandable body to the distal section of the elongated body.

19. The catheter of claim 18 wherein the deployment support member is coupled to an inner body positioned within the elongated body.

20. The catheter of claim 1 wherein the expandable body is manufactured from a shape memory alloy.

21. The catheter of claim 1 further comprising an atraumatic tip located at the distal section of the at least two support arms.

22. The catheter of claim 21 wherein the atraumatic tip comprises at least one end selected from the group consisting of a rounded end, a tapered end, and blunted end.

23. The catheter of claim 1 wherein the at least one thermal sensor is selected from the group consisting of thermal sensors, blood temperature sensors, and infrared sensors.

24. The catheter of claim 1 further comprising at least one ancillary thermal sensor located on at least one of the proximal section of the at least two support arms, the medial section of the at least two support arms, the distal section of the at least two support arms, the expandable body, and the elongated body.

25. The catheter of claim 24 wherein the at least one ancillary thermal sensor is selected from the group consisting of thermal sensors, blood temperature sensors, and, infrared sensors.

26. The catheter of claim 1 wherein the at least two support arms further comprises at least one sensor channel formed thereon and configured to receive the at least one sensor therein.

27. A catheter for measuring one or more characteristics of a body vessel, comprising:
an elongated body having a proximal end, a distal end, and a distal section;
an expandable body located at the distal section of the elongated body and comprising at least one support arm configured to be radially displaceable from and independently movable in relation to a longitudinal axis L of the expandable body and another support arm;
the at least one support arm having a proximal section configured to be radially displaceable a distance D1 from the longitudinal axis L, a medial section configured to be radially displaceable a distance D2 from the longitudinal axis L, and a distal section configured to be radially displaceable a distance D3 from the longitudinal axis L, wherein distance D2 is greater than distances D1 and D3; and
at least one sensor positioned on at least one of the proximal section of the at least one support arm, the medial section of the at least one support arm, the distal section of the at least one support arm, the expandable body, and the elongated body.

28. The catheter of claim 27 wherein the elongated body further comprises an inner body configured to couple to the expandable body.

29. The catheter of claim 28 wherein the inner body defines at least one internal passage therein.

30. The catheter of claim 29 wherein the at least one internal passage comprises a guidewire lumen configured to receive a guidewire therein.

31. The catheter of claim 28 further comprising an outer sleeve defining a sleeve lumen configured to receive at least one of the expandable body and the inner body therein.

32. The catheter of claim 31 wherein the outer sleeve is configured to controllably retract from the distal section of the elongated body.

33. The catheter of claim 31 wherein at least a portion of the inner body is configured to controllably extend beyond the outer sleeve in telescopic relation thereto.

34. The catheter of claim 30 further comprising a guidewire exit port positioned proximate to the distal section of the elongated body and in communication with the guidewire lumen formed in the at least one internal passage.

35. The catheter of claim 34 wherein the guidewire exit port is positioned about 5 cm to about 40 cm from the distal end of the elongated body.

36. The catheter of claim 27 further comprising a handle secured to the elongated body.

37. The catheter of claim 36 wherein the handle is detachably coupled to the elongated body.

38. The catheter of claim 36 further comprising an elongated body actuator positioned on the handle and located within an actuator recess formed on the handle.

39. The catheter of claim 38 wherein the elongated body actuator is in communication with at least one of an inner body formed within the elongated body and an outer sleeve positioned over the inner body.

40. The catheter of claim 36 further comprising a sensor cable secured to the handle and having a sensor connector secured thereto.

41. The catheter of claim 36 further comprising a proximal port formed on the handle and in communication with at least one of the elongated body and the expandable body.

42. The catheter of claim 27 wherein the expandable body is coupled to the distal section of the elongated body.

43. The catheter of claim 27 wherein the expandable body is secured to an inner body positioned within the elongated body.

44. The catheter of claim 27 further comprising a deployment support member wherein the expandable body is coupled to the distal section of the elongated body.

45. The catheter of claim 44 wherein the deployment support member is coupled to an inner body positioned within the elongated body.

46. The catheter of claim 27 wherein the expandable body is manufactured from at least one material selected from the group consisting of shape memory alloys, titanium, stainless steel, elgiloy, plastics, thermoplastics, elastomers, rubbers, silicones, and composite materials.

47. The catheter of claim 27 further comprising an atraumatic tip located at the distal section of the at least one support arm.

48. The catheter of claim 47 wherein the atraumatic tip comprises at least one end selected from the group consisting of a rounded end, a tapered end, and blunted end.

49. The catheter of claim 27 wherein the at least one sensor is selected from the group consisting of ultrasonic sensors, flow sensors, thermal sensors, blood temperature sensors, electrical contact sensors, conductivity sensors, electromagnetic detectors, chemical sensors, pH sensors, and infrared sensors.

50. The catheter of claim 27 further comprising at least one ancillary sensor located on at least one of the proximal section of the at least one support arm, the medial section of the at least one support arm, the distal section of the at least one support arm, the expandable body, and the elongated body.

51. The catheter of claim 50 wherein the at least one ancillary sensor is selected from the group consisting of ultrasonic sensors, flow sensors, thermal sensors, blood temperature sensors, electrical contact sensors, conductivity sensors, electromagnetic detectors, chemical sensors, pH sensors, and infrared sensors.

52. The catheter of claim 27 wherein the at least one support arm further comprises at least one sensor channel formed thereon and configured to receive the at least one sensor therein.

53. A catheter, comprising:
an elongated body having a proximal end, a distal end, and a distal section;
an expandable body located at the distal section of the elongated body and comprising at least one support arm;
at least one support arm retainer positioned on the distal section of the elongated body, the at least one support arm retainer independently securing at least one section of the at least one support arm to the elongated body in independently movable relation to another support arm and the elongated body; and
at least one sensor positioned on at least one of the distal section, the expandable body, and the at least one support arm.

54. The catheter of claim 53 wherein the elongated body further comprises an inner body configured to couple to the expandable body.

55. The catheter of claim 53 wherein the inner body defines at least one internal passage therein.

56. The catheter of claim 55 wherein the at least one internal passage comprises a guidewire lumen configured to receive a guidewire therein.

57. The catheter of claim 54 further comprising an outer sleeve defining a sleeve lumen configured to receive at least one of the expandable body and the inner body therein.

58. The catheter of claim 57 wherein the outer sleeve is configured to controllably retract from the distal section of the elongated body.

59. The catheter of claim 57 wherein at least a portion of the inner body is configured to controllably extend beyond the outer sleeve in telescopic relation thereto.

60. The catheter of claim 56 further comprising a guidewire exit port positioned proximate to the distal section of the elongated body and in communication with the guidewire lumen formed in the at least one internal passage.

61. The catheter of claim 60 wherein the guidewire exit port is positioned about 5 cm to about 40 cm from the distal end of the elongated body.

62. The catheter of claim 53 further comprising a handle secured to the elongated body.

63. The catheter of claim 62 wherein the handle is detachably coupled to the elongated body.

64. The catheter of claim 62 further comprising an elongated body actuator positioned on the handle and located within an actuator recess formed on the handle.

65. The catheter of claim 64 wherein the elongated body actuator is in communication with at least one of an inner body formed within the elongated body and an outer sleeve positioned over the inner body.

66. The catheter of claim 62 further comprising a sensor cable secured to the handle and having a sensor connector secured thereto.

67. The catheter of claim 62 further comprising a proximal port formed on the handle and in communication with at least one of the elongated body and the expandable body.

68. The catheter of claim 53 wherein the expandable body is coupled to the distal section of the elongated body.

69. The catheter of claim 53 wherein the expandable body is secured to an inner body positioned within the elongated body.

70. The catheter of claim 67 wherein the expandable body is secured to an inner body located within the elongated body with at least one support arm retainer.

71. The catheter of claim 53 wherein the expandable body further comprises a deployment support member configured to be secured to the elongated body.

72. The catheter of claim 71 wherein the deployable support member is secured to the elongated body with an adhesive.

73. The catheter of claim 71 wherein five support arms are rigidly attached to the deployment support member.

74. The catheter of claim 53 wherein the expandable body is manufactured from a shape memory alloy.

75. The catheter of claim 53 further comprising at least one sensor coupled to the at least one support arm.

76. The catheter of claim 75 wherein the at least one sensor is adhesively coupled to the at least one support arm.

77. The catheter of claim 75 further comprising at least one sensor slot formed on the at least one support arm and configured to receive the at least one sensor therein.

78. The catheter of claim 53 further comprising at least one retaining device positioned on the at least one support arm and configured to movably secure the at least one support arm to the at least one support arm retainer.

79. The catheter of claim 53 further comprising at least one flex member secured to the at least one support arm and configured to permit a radial and longitudinal flexing of the at least one support arm.

80. The catheter of claim 53 further comprising an expandable body passage formed within the expandable body.

81. The catheter of claim 80 further comprising a guidewire lumen traversing the expandable body passage and in communication with a guidewire exit port formed on the elongated body and a guidewire port formed on the distal section, the guidewire lumen configured to receive a guidewire therein.

82. The catheter of claim 81 further comprising the at least one support arm retainer secured to at least one the guidewire lumen and the distal section.

83. The catheter of claim 82 wherein the at least one support arm retainer is adhesively attached to at least one the guidewire lumen and the distal section.

84. The catheter of claim 53 wherein the at least one support arm retainer comprises a support arm channel and configured to movably receive a support arm body of the at least one support arm therein.

85. The catheter of claim 53 further comprising a retainer sleeve capable of coupling to the distal section of the elongated body and configured to receive the at least one support arm retainer therein.

86. The catheter of claim 53 wherein the at least one support arm retainer comprises a support arm retaining sleeve positioned on at least one of the elongated body, the distal section and sized to receive and retain at least a section of the at least one support arm therein.

87. The catheter of claim 53 further comprising a distal tip coupled to the expandable body.

88. The catheter of claim 87 further comprising at least one support arm retainer secured to the distal tip.

89. The catheter of claim 87 further comprising a distal tip coupled to the expandable body and disposing a guidewire port thereon, the guidewire port in communication with the elongated body through a guidewire lumen positioned within the expandable body.

90. The catheter of claim 87 further comprising a distal tip coupled to the expandable body and disposing a guidewire port thereon, the guidewire port in communication with the elongated body through a guidewire lumen positioned within the expandable body, the guidewire lumen in communication with a guidewire exit port located on the elongated body.

91. The catheter of claim 90 wherein the guidewire exit port is positioned about 5 cm to about 40 cm from the distal end of the elongated body.

92. The catheter of claim 53 wherein the at least one sensor comprises at least one sensor selected from the group consisting of ultrasonic sensors, flow sensors, thermal sensors, blood temperature sensors, electrical contact sensors, conductivity sensors, electromagnetic detectors, chemical sensors, pH sensors, and infrared sensors.

93. The catheter of claim 53 further comprising at least one ancillary sensor located on at least one of the at least one support arm, the expandable body, and the elongated body.

94. The catheter of claim 93 wherein the at least one ancillary sensor is selected from the group consisting of ultrasonic sensors, flow sensors, thermal sensors, blood temperature sensors, electrical contact sensors, conductivity sensors, electromagnetic detectors, chemical sensors, pH sensors, and infrared sensors.

95. A catheter, comprising:
an elongated body having a proximal end, a distal end, and a distal section and having an inner body member positioned within a retractable outer sleeve;
an expandable body located at the distal section of the elongated body and positionable within the outer sleeve, the expandable body comprising at least one support arm coupled proximately to the elongated body in fixed relation;
at least one support arm retainer positioned on the distal section of the elongated body which independently couples a distal section of the at least one support arm to the distal section of the elongated body in independently movable relation to each of the other at least one support arms; and
at least one thermal sensor positioned on at least one of the distal section, the expandable body, the deployment support member, and the at least one support arm.

96. The catheter of claim 95 wherein the sleeve lumen further comprises a inner body positioned therein and defining an internal passage capable of receiving a guidewire therein.

97. The catheter of claim 96 further comprising:
a guidewire port formed on the distal section of the elongated body and in communication with the internal passage of the inner body and capable of receiving a guidewire therein; and
a guidewire exit slot formed on the movable outer sleeve and in communication with the guidewire port through the internal passage of the inner body, the guidewire exit slot capable of receiving the guidewire therethrough.

98. The catheter of claim 97 wherein the guidewire exit port is positioned about 5 cm to about 40 cm from the distal end of the elongated body.

99. The catheter of claim 96 further comprising a guidewire port formed on the distal section of the elongated body and in communication with the internal passage of the inner body.

100. The catheter of claim 95 further comprising a handle secured to the elongated body.

101. The catheter of claim 100 wherein the elongated body is detachably coupled to the handle.

102. The catheter of claim 100 further comprising an elongated body actuator positioned on the handle in movable relation to the handle and in communication with the outer sleeve.

103. The catheter of claim 100 further comprising a proximal port positioned on the handle and in fluid communication with the elongated body.

104. The catheter of claim 103 wherein the proximal port is configured to receive a guidewire therein.

105. The catheter of claim 95 wherein the expandable body is manufactured from at least one material selected from the group consisting of shape memory alloys, stainless steel, titanium, elgiloy, polymers, plastic, thermoplastics, and composite materials.

106. The catheter of claim 95 further comprising at least one sensor slot formed on the at least one support arm and configured to receive the at least one sensor therein.

107. The catheter of claim 95 further comprising at least one retaining device positioned on the at least one support arm and configured to movably secure the at least one support arm to the at least one support arm retainer.

108. The catheter of claim 95 further comprising at least one flex member secured to the at least one support arm and configured to permit a radial and longitudinal flexing of the at least one support arm.

109. The catheter of claim 97 further comprising an expandable body passage formed within the expandable body;

110. The catheter of claim 109 further comprising a guidewire lumen traversing the expandable body passage and coupled to at least one of the internal passage, the guidewire exit port, and to a guidewire port formed on the distal section of the elongated body, the guidewire lumen configured to receive a guidewire therein.

111. The catheter of claim 110 further comprising the at least one support arm retainer secured to the guidewire lumen.

112. The catheter of claim 111 wherein the at least one support arm retainer is adhesively attached to the guidewire lumen.

113. The catheter of claim 95 further comprising a distal tip coupled to the expandable body.

114. The catheter of claim 113 further comprising at least one support arm retainer secured to the distal tip.

115. The catheter of claim 110 further comprising a distal tip coupled to the expandable body and disposing the guidewire port thereon, the guidewire port in coupled to the guidewire lumen.

116. The catheter of claim 95 wherein the at least one support arm retainer comprises a support arm channel and configured to movably receive a support arm body of the at least one support arm therein.

117. The catheter of claim 95 further comprising a retainer sleeve capable of coupling to the distal section of the elongated body and configured to receive the at least one support arm retainer therein.

118. The catheter of claim 95 wherein the at least one sensor comprises at least one sensor selected from the group consisting of ultrasonic sensors, flow sensors, thermal sensors, blood temperature sensors, electrical contact sensors, conductivity sensors, electromagnetic detectors, chemical sensors, and infrared sensors.

119. A deployable expandable body for measuring one or more thermal characteristics of a body vessel, comprising:
at least two support arms configured to be radially displaceable from a longitudinal axis of the expandable body and independently movable in a radial direction in relation to each other when deployed;
the support arms having a proximal section, a distal section, and a medial section configured to be radially displaceable a greater distance from the longitudinal axis than the proximal and distal sections; and
a thermal sensor disposed on the medial section of at least one of the support arms and configured to contact the body vessel when deployed.

120. An expandable body for measuring one or more thermal characteristics of a body vessel, comprising:
at least two deployable support arms configured to be radially displaceable from a longitudinal axis of the expandable body and independently movable in relation to each other when deployed;
the at least two support arms having a proximal section configured to be radially displaceable a distance D1 from the longitudinal axis L, a medial section configured to be radially displaceable a distance D2 from the longitudinal axis L, and a distal section configured to be radially displaceable a distance D3 from the longitudinal axis L, wherein distance D2 is greater than distances D1 and D3; and
at least one sensor positioned on the medial section of the at least two support arms.

121. The expandable body of claim 120 wherein the expandable body is manufactured from at least one material selected from the group consisting of shape memory alloys, Nitinol, stainless steel, titanium, elgiloy, polymers, plastic, thermoplastics, and composite materials.

122. The expandable body of claim 120 further comprising at least one sensor slot formed on the at least two support arms, the sensor slot configured to receive the at least one sensor therein.

123. An expandable body for measuring one or more thermal characteristics of a vessel, comprising:
at least one support arm configured to be radially displaceable from and independently movable in relation to a longitudinal axis L of the expandable body and another support arm;
the at least one support arm having a proximal section configured to be radially displaceable a distance D1 from the longitudinal axis L, a medial section configured to be radially displaceable a distance D2 from the longitudinal axis L, and a distal section configured to be radially displaceable a distance D3 from the longitudinal axis L, wherein distance D2 is greater than distances D1 and D3; and
at least one sensor positioned on at least one of the proximal section of the at least one support arm, the medial section of the at least one support arm, the distal section of the at least one support arm, and the expandable body.

124. The expandable body of claim 123 wherein the expandable body is manufactured from at least one material selected from the group consisting of shape memory alloys, Nitinol, stainless steel, titanium, elgiloy, polymers, plastic, thermoplastics, and composite materials.

125. The expandable body of claim 123 further comprising at least one sensor slot formed on the at least two support arms, the sensor slot configured to receive the at least one sensor therein.

126. An expandable body, comprising:
at least one support arm defining the expandable body;
at least one support arm retainer configured to retain at least a section of the at least one support arm therein in independently movable relation to at least another support arm; and
at least one sensor positioned on at least one of the at least support arm and the at least one support arm retainer.

127. An expandable body, comprising:
at least one support arm defining the expandable body, the at least one support arm having a sensor channel formed thereon;
at least one support arm retainer configured to retain at least a section of the at least one support arm therein in independently movable relation to at least another support arm; and
at least one sensor positioned within the sensor channel of the at least one support arm.

128. An expandable body, comprising:
at least one support arm defining an expandable body having a longitudinal axis L, the at least one support arm having a first relaxed state displaced a first distance D1 from the longitudinal axis L and a second compressed state displaced a second distance D2 from the longitudinal axis L, wherein the first distance D1 is greater than the second distance D2;
at least one support arm retainer configured to retain at least a section of the at least one support arm therein in independently movable relation to at least another support arm; and
at least one sensor positioned on at least one of the at least one support arm and the at least one support arm retainer.

129. A catheter, comprising:
an elongated body having a distal section;
an expandable body defining an expandable body passage disposed on the distal section of the elongated body;
at least one thermal sensor configured to contact a vessel wall disposed on the expandable body; and
an inner body having a flexible distal section co-linear with the expandable body.

130. The catheter of claim 129 wherein the flexible distal section of the inner body further comprising a guidewire lumen disposed within the expandable body passage.

131. The catheter of claim 130 wherein the flexible distal section of the inner body further comprises a flexible coiled member positioned within an expandable body passage and defining a coil passageway sized to receive a guidewire therein.

132. The catheter of claim 131 wherein the coiled member is secured to the elongated body and traverses the expandable body passage.

133. The catheter of claim 131 wherein the coiled member partially traverses the expandable body passage.

134. A catheter, comprising:
an elongated body having a distal section;
an expandable body defining an expandable body passage disposed on the distal section of the elongated body;
a flexible distal tip disposed on the expandable body and partially traversing the expandable body passage;
at least one thermal sensor configured to contact a vessel wall disposed on the expandable body; and
an inner body having a flexible distal section co-linear with the expandable body.

135. A catheter, comprising:
an elongated body having a distal section;
an expandable body defining an expandable body passage disposed on the distal section of the elongated body;
a coiled member defining a coil passageway coupled to the elongated body and traversing the expandable body passage;
at least one thermal sensor configured to contact a vessel wall disposed on the expandable body; and
an inner body having a flexible distal section co-linear with the expandable body.
